SEQUENCE LISTING (14 sequences)

SEQ ID NO: 1 - DNA, 36 bp, Artificial Sequence
  Synthetic Hsp90 mutagenesis primer
gaaagcttgacagatcccagtcaattagactctggg

SEQ ID NO: 2 - DNA, 37 bp, Artificial Sequence
  Synthetic Hsp90 mutagenesis primer
gaaagcttgacagatcccagtagattagactctggga

SEQ ID NO: 3 - DNA, 36 bp, Artificial Sequence
  Synthetic Hsp90 mutagenesis primer
gatactggaattggaatgacccaggctgacttgatc

SEQ ID NO: 4 - DNA, 36 bp, Artificial Sequence
  Synthetic Hsp90 mutagenesis primer
gatactggaattggaatgaccagggctgacttgatc

SEQ ID NO: 5 - DNA, 37 bp, Artificial Sequence
  Synthetic Hsp90 mutagenesis primer
tcgatcaagaagagctcaaccaaacaaagcccatctg

SEQ ID NO: 6 - DNA, 37 bp, Artificial Sequence
  Synthetic Hsp90 mutagenesis primer
tcgatcaagaagagctcaacagaacaaagcccatctg

SEQ ID NO: 7 - DNA, 37 bp, Artificial Sequence
  Synthetic Hsp90 mutagenesis primer
tgggaagatcacttggcagtgcagcatttttcagttg

SEQ ID NO: 8 - DNA, 37 bp, Artificial Sequence
  Synthetic Hsp90 mutagenesis primer
tgggaagatcacttggcagtgaggcatttttcagttg

SEQ ID NO: 9 - DNA, 37 bp, Artificial Sequence
  Synthetic Hsp90 mutagenesis primer
gtgatgagatggtttctctccaggactactgcaccag

SEQ ID NO: 10 - DNA, 37 bp, Artificial Sequence
  Synthetic Hsp90 mutagenesis primer
gtgatgagatggtttctctcagggactactgcaccag

SEQ ID NO: 11 - DNA, 39 bp, Artificial Sequence
  Synthetic Hsp90 mutagenesis primer
gaagactttagtgtcagtcacccaagaaggcctggaact

SEQ ID NO: 12 - DNA, 39 bp, Artificial Sequence
  Synthetic Hsp90 mutagenesis primer
gaagactttagtgtcagtcaccagagaaggcctggaact

SEQ ID NO: 13 - DNA, 35 bp, Artificial Sequence
  Synthetic Hsp90 mutagenesis primer
tccagaggatgaagaagagcaaaagaagcaggaag

SEQ ID NO: 14 - DNA, 35 bp, Artificial Sequence
  Synthetic Hsp90 mutagenesis primer
tccagaggatgaagaagagagaaagaagcaggaag

(19) United States
(12) Patent Application Publication
(10) Pub. No.: US 2010/0111943 A1
(43) Pub. Date: May 6, 2010

(54) COMPOSITIONS AND METHODS FOR INHIBITING CANCER METASTASIS

(75) Inventors: Kapil Bhalla, Martinez, GA (US); Yonghua Yang, Chapel Hill, NC (US)

Correspondence Address: Pabst Patent Group LLP, 1545 PEACHTREE STREET NE, SUITE 320, ATLANTA, GA, 30309, US

(73) Assignee: Medical College of Georgia Research Institute, Inc

(21) Appl. No.: 12/531,973

(22) PCT Filed: Mar. 24, 2008
(86) PCT No.: PCT/US2008/058021

§ 371 Date: Sep. 18, 2009

Related U.S. Application Data
(60) Provisional application No. 60/919,484, filed on Mar. 22, 2007.

(51) Int. Cl. A61K 39/395; A61K 38/16; A61K 38/43; C12N 9/00; C07K 16/00; C12N 5/00; A61K 31/7088; C07H 21/00

(52) U.S. Cl. 424/133.1; 424/130.1; 514/12; 424/141.1; 424/94.1; 435/183; 530/387.3; 530/388.1; 435/325; 514/44 R; 536/23.1

(57) ABSTRACT

It has been discovered that antagonists of acetylated heat shock proteins can inhibit or reduce tumor cell invasion or metastasis. Compositions and methods for inhibiting tumor cell invasion or metastasis are provided. One embodiment provides a pharmaceutical composition including a heat shock protein antagonist in an amount effective to inhibit or reduce tumor cell invasion or metastasis. Another embodiment provides a pharmaceutical composition including a heat shock protein deacetylase in an amount effective to inhibit or reduce secretion of heat shock proteins. Representative target heat shock proteins include, but are not limited to hsp90α and hsp70. Methods of treating cancer or inhibiting tumor cell invasion and metastasis are also provided.

### CROSS-REFERENCE TO RELATED APPLICATIONS

This application claims priority to and benefit of U.S. Patent No. 60/919,484 filed on Mar. 22, 2007, and where permissible is incorporate by reference in its entirety.

### FIELD OF THE INVENTION

The invention is generally directed to pharmaceutical compositions and methods for treating cancer and inhibiting or reducing metastasis.

### BACKGROUND OF THE INVENTION

Cancer has an enormous physiological and economic impact. For example a total of 1,437,180 new cancer cases and 565,650 deaths from cancer are projected to occur in the United States in 2008 (Jemal, A., Cancer J Clin, 58:71-96 (2008)). The National Institutes of Health estimate overall costs of cancer in 2007 at $219.2 billion: $89.0 billion for direct medical costs (total of all health expenditures); $18.2 billion for indirect morbidity costs (cost of lost

productivity due to illness); and $112.0 billion for indirect mortality costs (cost of lost productivity due to premature death). Although many cancer therapies are available, they are usually associated with adverse side-effects. More effective treatments and treatments with fewer side effects are needed.

Heat shock proteins are versatile molecular chaperones involved in many cellular functions including proper folding, assembly of multiunit complexes, activation, and transport of proteins (Eustance, B. K., Jay, Daniel, Cell Cycle, 3(9):1098-1100 (2004). Apart from their intracellular location, heat shock proteins with a molecular weight of 70 and 90 kDa have been found on the plasma membrane of malignantly transformed cells (Sherman and Multhoff, Ann. N.Y. Acad. Sci., 1113: 192-201 (2007); Eustace, B. K., et al. Nat. Cell Biol., 6(6):507-14 (2004)). Because of their role in several cell functions, a large amount of research has been conducted on them including developing heat shock protein antagonists for the treatment of cancer.

Antagonists of heat shock proteins include geldanamycin (“GDA”), a macrocyclic lactam that is a member of the benzoquinone-containing ansamycins family of natural products. The isolation, preparation and various uses of geldanamycin are described in U.S. Pat. No. 3,595,955. Like most naturally-occurring members of this class of molecules, geldanamycin is typically produced as a fermentation product of Streptomyces hygroscopicus var. geldanus var. nova strain (DeBoer, C. et al., Journal of Antibiotics, 23:442-447 (1970)). Other analogs and derivatives of geldanamycin have been identified or synthesized, and their use as anti-tumor agents is described in U.S. Pat. Nos. 7,259,156; 7,208,630; 7,026,350; and 6,890,917 as well as in several others. One member of this family that has been examined in some detail is 17-allylamino-17-demethoxygeldanamycin (“17-AAG”).

Additional known inhibitors of Hsp90 include the anti-tumor antibiotics geldanamycin (“GDA”), radicicol (“RDC”), herbimycin A (“HB”), a 17-allylamino derivative of GDA (“17-AAG”), and the synthetic ATP analog called PU3. These inhibitors exert their activity by binding to the N-terminal ATP binding pocket and inhibit the ATPase activity of Hsp90. The energy normally derived from ATP hydrolysis is used to elicit a conformational change that releases the properly folded client protein from Hsp90. However, when a non-hydrolyzable inhibitor is present, Hsp90 is unable to fold the bound client protein, resulting in ubiquitination of the client protein and subsequent proteolysis by the proteasome.

Tumor cell invasiveness is crucial for cancer metastasis and is not yet understood. The hsp90 alpha isoform, but not hsp90 beta, is expressed extracellularly where it interacts with the matrix metalloproteinase 2 (MMP2). Inhibition of extracellular hsp90 alpha decreases both MMP2 activity and invasiveness (Eustace, B. K., et al. Nat Cell Biol., 6(6):507-14 (2004) Epub 2004, May 16). Small molecule cell-impermeant Hsp90 antagonists inhibit tumor cell motility and invasion by interfering with leading edge actin polymerization and focal adhesion formation (Tsutsumi, S. et al., Oncogene, 1-10 (2007)). Although heat shock protein inhibitors are known in the art, inhibitors with a higher degree of specificity and efficacy are needed.

Therefore, it is an object of the invention to provide compositions and methods for inhibiting the secretion of heat shock proteins.

It is another object of the invention to provide compositions and methods for treating cancer.

It is still another object to provide compositions and methods for inhibiting the acetylation of heat shock proteins.

It is another object to provide compositions and methods for inhibiting metastasis.

It is another object to provide compositions and methods for inhibiting tumor cell invasion.

### SUMMARY OF THE INVENTION

It has been discovered that antagonists of acetylated heat shock proteins can inhibit or reduce tumor cell invasion or metastasis. Compositions and methods for inhibiting tumor cell invasion or metastasis are provided. One embodiment provides a pharmaceutical composition including a heat shock protein antagonist in an amount effective to inhibit or reduce tumor cell invasion or metastasis. Another embodiment provides a pharmaceutical composition including a heat shock protein deacetylase in an amount effective to inhibit or reduce secretion of heat shock proteins. Representative target heat shock proteins include, but are not limited to hsp90α and hsp70.

Another embodiment provides methods for inhibiting tumor cell invasion or metastasis by administering a heat shock protein antagonist. Preferably, the heat shock antagonist specifically binds to acetylated heat shock proteins and inhibits or reduces acetylated heat shock protein biological activity. In one embodiment, the heat shock protein antagonist inhibits or reduces the ability of heat shock proteins from promoting or activating matrix metalloprotein-2 (MMP-2) activity.

Still another embodiment provides methods for inhibiting the secretion of heat shock proteins or the translocation of heat shock proteins to the extracellular surface of the cell by administering to a subject an effective amount of a heat shock protein deacetylase. Methods for inhibiting tumor cell invasion or metastasis by administering an effective amount of a heat shock protein deacetylase are also provided.

Another embodiment provides methods for identifying inhibitors of acetylated heat shock proteins.

### BRIEF DESCRIPTION OF THE DRAWINGS

FIG. 1A is bar graph showing percent invasion of MB-231 cells treated with 10, 20, 40 nM LBH589. FIG. 1B is a bar graph of showing percent invasion of MB-486 cells treated with 10 or 20 nM LBH589 or 0.5 or 1.0 μM vorinostat. FIG. 1C is a bar graph showing percent invasion of MB-486 cells transfected with the indicated mutant hsp90α.

FIG. 2A is bar graph of percent intensity of immunoprecipitation of extracellular or intracellular hsp90α or the K69Q mutant thereof. FIG. 2B is a bar graph showing percent invasion of MB-231 cells treated with 20 μg of anti-hsp90 antibody or AcK antibody.

### DETAILED DESCRIPTION OF THE INVENTION

### I. Definitions

Unless otherwise defined, all technical and scientific terms used herein have the same meaning as commonly understood by one of ordinary skill in the art to which this invention pertains. All patent publications, patent applications, and patents mentioned herein are incorporated by reference in their entirety. In case of conflict, the present specification, including definitions, will control. In addition, the materials, methods, and examples are illustrative only and not intended to be limiting.

The term “effective amount” or “therapeutically effective amount” with regard to cancer means a dosage sufficient to reduce, prevent, or inhibit one or more symptoms associated with cancer or to otherwise provide a desired pharmacologic and/or physiologic effect. These terms can also be used with regard to acetylation of heat shock protein function or degree of acetylation. The precise dosage will vary according to a variety of factors such as subject-dependent variables (e.g., age, immune system health, etc.), the disease, and the treatment being effected.

The terms “individual,” “individual,” “subject,” and “patient” are used interchangeably herein, and refer to a mammal, including, but not limited to, rodents, simians, humans, mammalian farm animals, mammalian sport animals, and mammalian pets.

The term “heat shock protein antagonist” means a substance that interferes with, inhibits, blocks or reduces heat shock protein biological function, in particular extracellular heat shock protein function, or acetylation of heat shock proteins, or secretion of heat shock proteins. A representative heat shock protein biological function includes, but is not limited to activating matrix metalloproteinase-2 activity. Inhibition and reduction are relative to a control.

### I. Compositions

Compositions for inhibiting or reducing tumor cell invasion and/or metastasis are provided. Preferred compositions are those that interfere, inhibit, reducer block extracellular heat shock protein function, in particular acetylated heat shock protein function. In one embodiment the composition includes an antagonist of hsp90α, hsp70, or a combination thereof. A preferred heat shock protein antagonist includes, but is not limited to an antibody that binds an acetylated amino acid of a heat shock protein, for example hsp90α or hsp70.

Other embodiments provide compositions for inhibiting or reducing acetylation of heat shock proteins, preferably hsp90α or hsp70. A representative composition that inhibits or reduces acetylation of heat shock protein is a deacetylase, preferably histone deacetylase.

Another embodiment is directed to compositions comprising a heat shock antagonist in an amount effective to inhibit or reduce MMP-2 activity relative to a control. It will be appreciated that a control includes cells or organisms that are not treated with the disclosed compositions.

In a preferred embodiment, the heat shock protein antagonist includes an antibody that specifically binds to the heat shock protein and inhibits or reduces one or more biological functions of the heat shock protein. Preferably the antibody inhibits or reduces the ability of the heat shock protein to promote MMP-2 activity. The antibody can be a single chain, humanized, chimeric, monoclonal, or a polyclonal antibody or an antigen binding fragment thereof. In one embodiment, the heat shock protein antagonist is a diabody. It will be appreciated that the heat shock protein antagonist can be any substance or compound that selectively binds to heat shock proteins, in particular to acetylated heat shock proteins. Such substances can include small molecules, polypeptides, or nucleic acids, i.e., aptamers. In one embodiment, the heat shock protein antagonist selectively antagonizes extracellular heat shock proteins over intracellular heat shock proteins.

In another embodiment, the disclosed heat shock protein antagonists selectively bind to an acetylated amino acid of the heat shock protein. Preferred acetylated amino acids of hsp90α include, but are not limited to K69, K100, K292, K327, K478, K546 and K558, or a combination thereof.

The compositions are administered to a individual in need of treatment or prophylaxis of at least one symptom or manifestation (since disease can occur/progress in the absence of symptoms) of cancer or cellular hyperproliferation. In one embodiment, the compositions are administered in an effective amount to inhibit acetylated heat shock protein activation of MMP2 and thereby inhibit or reduce tumor cell invasion and/or metastasis. The amount of inhibition acetylated heat shock protein can be determined relative to a control, for example cells that are not treated with the inhibitor. Methods for measuring inhibition of acetylation of heat shock proteins are provided in the Examples.

A. Formulations

The compounds are preferably employed for therapeutic uses in combination with a suitable pharmaceutical carrier. Such compositions include an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are described herein.

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compound alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. For administration by inhalation, the compounds are conveniently delivered in the form of an aerosol spray presentation from pressurized packs or a nebulizer, with the use of a suitable propellant.

In some embodiments, the compound described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the compounds are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett. 14(19):4975-4977 (2004)) and in viva (Soutschek, et al., Nature 432(7014):173-178 (2004)). Other groups that can be attached or conjugated to the compounds described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; enzymes such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also described methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

B. Methods of Administration

In general, methods of administering compounds are well known in the art. The compositions can be administered by a number of routes including, but not limited to: oral, intravenous, intraperitoneal, intramuscular, transdermal, subcutaneous, topical, sublingual, or rectal means. Compounds can also be administered via liposomes. Such administration routes and appropriate formulations are generally known to those of skill in the art.

Administration of the formulations described herein may be accomplished by any acceptable method which allows the compounds to reach its target. The particular mode selected will depend of course, upon factors such as the particular formulation, the severity of the state of the subject being treated, and the dosage required for therapeutic efficacy. As generally used herein, an “effective amount” is that amount which is able to treat one or more symptoms of age related disorder, reverse the progression of one or more symptoms of age related disorder, halt the progression of one or more symptoms of age related disorder, or prevent the occurrence of one or more symptoms of age related disorder in a subject to whom the formulation is administered, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intradermally for treatment or prevention of age related disorder, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The formulations may be delivered using a bioerodible implant by way of diffusion or by degradation of the polymeric matrix. In certain embodiments, the administration of the formulation may be designed so as to result in sequential exposures to the active agent over a certain time period, for example, hours, days, weeks, months or years. This may be accomplished, for example, by repeated administrations of a formulation or by a sustained or controlled release delivery system in which the active agent is delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the heat shock protein antagonist is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the heat shock protein antagonist. In addition, a pump-based hardware delivery system may be used to deliver one or more embodiments.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

C. Effective Dosages

Dosages for a particular individual can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the heat shock protein antagonist employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

Formulations are administered at a rate determined by the LD50 of the relevant formulation, and/or observation of any side-effects of the compositions at various concentrations, e.g., as applied to the mass and overall health of the individual. Administration can be accomplished via single or divided doses.

In vitro models can be used to determine the effective doses of the compositions as a potential cancer treatment, as described in the examples. In determining the effective amount of the compound to be administered in the treatment or prophylaxis of disease the physician evaluates circulating plasma levels, formulation toxicities, and progression of the disease. For the disclosed compositions, the dose administered to a 70 kilogram individual is typically in the range equivalent to dosages of currently-used therapeutic antibodies such as Avastin®, Erbitux® and Herceptin®.

The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, the compositions can also be administered in therapeutically effective amounts as a portion of an anti-cancer cocktail. Anti-cancer cocktails can include therapeutics to treat cancer or angiogenesis of tumors.

### II. Methods of Treatment

The disclosed compositions can be administered to a subject in need thereof to treat, alleviate, or reduce one or more symptoms associated with cancer or other forms of cellular hyperproliferation. The compositions can be administered locally or systemically to inhibit tumor cell invasion or tumor cell metastasis. The types of cancer that can be treated with the provided compositions and methods include, but are not limited to, the following: bladder, brain, breast, cervical, colorectal, esophageal, kidney, liver, lung, nasopharangeal, pancreatic, prostate, skin, stomach, uterine, ovarian, and testicular. Administration is not limited to the treatment of an existing tumor but can also be used to prevent or lower the risk of developing such diseases in an individual, i.e., for prophylactic use. Potential candidates for treatment include individuals with a high risk of developing cancer, i.e., with a personal or familial history of certain types of cancer.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

### III. Methods for Screening for Inhibitors of Tumor Cell Invasion and Metastasis

Methods for identifying inhibitors of tumor cell invasion or metastasis are provided and utilize well known techniques and reagents. The inhibitor reduces, inhibits, blocks, or interferes with heat shock protein function, expression, or bioavailability. Preferred heat shock proteins include, but are not limited to hsp90α and hsp70. Preferred inhibitors reduce or inhibit acetylation of heat shock proteins, or the function of acetylated heat shock proteins. Other preferred inhibitors are those that inhibit the secretion of heat shock proteins or inhibit the translocation of heat shock proteins to the exterior cell surface.

In some embodiments, the assays can include random screening of large libraries of test compounds. The test compounds are typically, non-protein small molecules. The term “small molecule” refers to compounds less than 1,000 daltons, typically less than 500 daltons. Alternatively, the assays may be used to focus on particular classes of compounds suspected of inhibiting acetylation of heat shock proteins or secretion of heat shock proteins in cells, tissues, organs, or systems.

Assays can include determinations of heat shock protein expression, protein expression, protein activity, signal transduction, or binding activity. Other assays can include determinations of heat shock protein nucleic acid transcription or translation, for example mRNA levels, mRNA stability, mRNA degradation, transcription rates, and translation rates.

In one embodiment, the identification of an inhibitor of tumor cell invasion or metastasis is based on the function of heat shock protein in the presence and absence of a test compound. The test compound or modulator can be any substance that alters or is believed to alter the function of heat shock protein, in particular the acetylation of heat shock protein, secretion of heat shock protein, or relocation of heat shock protein to the exterior cell surface. Typically, an inhibitor will be selected that reduces, eliminates, or inhibits extracellular heat shock protein function, acetylation of heat shock proteins, or the secretion of heat shock proteins.

One exemplary method includes contacting heat shock protein with at least a first test compound, and assaying for an interaction between the heat shock protein and the first test compound with an assay. The assaying can include determining inhibition of heat shock protein acetylation, secretion, or activation of matrix metalloproteinase-2 (MMP2).

Specific assay endpoints or interactions that may be measured in the disclosed embodiments include assaying for heat shock protein acetylation, secretion, MMP2 activity, modulation, down or up regulation or turnover. These assay endpoints may be assayed using standard methods such as FACS, FACE, ELISA, Northern blotting and/or Western blotting. Moreover, the assays can be conducted in cell free systems, in isolated cells, genetically engineered cells, immortalized cells, or in organisms such as transgenic animals.

Other screening methods include using labeled heat shock protein to identify a test compound. Heat shock protein can be labeled using standard labeling procedures that are well known and used in the art. Such labels include, but are not limited to, radioactive, fluorescent, biological and enzymatic tags.

Another embodiment provides a method for identifying an inhibitor of tumor cell invasion or metastasis by determining the effect a test compound has heat shock protein acetylation, secretion of MMP2 activity. For example isolated cells or whole organisms expressing heat shock proteins or both can be contacted with a test compound. Heat shock protein secretion, acetylation, and MMP2 activity can be determined using standard biochemical techniques such as immunodetection. Suitable cells for this assay include, but are not limited to, cancer cells, immortalized cell lines, primary cell culture, or cells engineered to express specific heat shock proteins, for example cells from mammals such as humans. Compounds that inhibit heat shock protein activation of MMP2, heat shock protein secretion, or heat shock protein acetylation or a combination thereof can be selected.

Another embodiment provides for in vitro assays for the identification of inhibitors of tumor cell invasion or metastasis. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule, for heat shock protein or acetylated heat shock protein, in a specific fashion is strong evidence of a related biological effect. Such a molecule can bind to an acetylated heat shock protein and inhibit its biological functions. The binding of a molecule to a target may, in and of itself, be inhibitory, due to steric, allosteric or charge—charge interactions or inactivation of acetylated heat shock protein. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

Other embodiments include methods of screening compounds for their ability to inhibit the function of acetylated heat shock proteins or the secretion of acetylated heat shock protein from cells. Various cell lines can be utilized for such screening assays, including cells specifically engineered for this purpose. Suitable cells include cancer cells that express heat shock proteins such as hsp90α or hsp70. Furthermore, those of skill in the art will appreciate that stable or transient transfections, which are well known and used in the art, may be used in the disclosed embodiments.

For example, a transgenic cell comprising an expression vector can be generated by introducing the expression vector into the cell. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome mediated transfection, DEAE dextran mediated transfection, electroporation, ballistic bombardment, and the like. Alternatively, cells may be simply transfected with the disclosed expression vector using conventional technology described in the references and examples provided herein. The host cell can be a prokaryotic or eukaryotic cell, or any transformable organism that is capable of replicating a vector and/or expressing a heterologous gene encoded by the vector. Numerous cell lines and cultures are available for use as a host cell, and they can be obtained through the American Type Culture Collection (ATCC), which is an organization that serves as an archive for living cultures and genetic materials (www.atcc.org).

A host cell can be selected depending on the nature of the transfection vector and the purpose of the transfection. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE, La Jolla, Calif.). Alternatively, bacterial cells such as E. coli LE392 could be used as host cells for phage viruses. Eukaryotic cells that can be used as host cells include, but are not limited to, yeast, insects and mammals. Examples of mammalian eukaryotic host cells for replication and/or expression of a vector include, but are not limited to, HeLa, NIH3T3, Jurkat, 293, Cos, CHO, Saos, and PC12. Examples of yeast strains include YPH499, YPH500 and YPH501. Many host cells from various cell types and organisms are available and would be known to one of skill in the art. Similarly, a viral vector may be used in conjunction with either an eukaryotic or prokaryotic host cell, particularly one that is permissive for replication or expression of the vector. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

In vivo assays involve the use of various animal models, including non-human transgenic animals that have been engineered to have specific defects, or carry markers that can be used to measure the ability of a test compound to reach and affect different cells within the organism. Due to their size, ease of handling, and information on their physiology and genetic make-up, mice are a preferred embodiment, especially for transgenic animals. However, other animals are suitable as well, including C. elegans, rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

Determining the effectiveness of a compound in vivo may involve a variety of different criteria. Also, measuring toxicity and dose response can be performed in animals in a more meaningful fashion than in in vitro or in cyto assays.

Unless defined otherwise, all technical and scientific terms used herein have the same meanings as commonly understood by one of skill in the art to which the disclosed invention belongs. Patent publications cited herein and the materials for which they are cited are specifically incorporated by reference.

Unless defined otherwise, all technical and scientific terms used herein have the same meanings as commonly understood by one of skill in the art to which the disclosed invention belongs. Patent publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Hyperacetylation of hsp90α Involves p300 as the Acetyl-Transferase

### Materials and Methods

Cell Lines, Antibodies and Plasmids:

HEK293T, MDA-MB-468, MDA-MB-231 and T47D cells were all purchased from American Tissue Culture Collection (Manassas, Va.). HEK293T and MDAMB-468 cells were maintained in Dulbecco' modified Eagle's medium (DMEM); T47D and MDAMB-231 cells were maintained in RPMI medium containing 10% FBS. The following antibodies used were purchased from commercial sources: anti-CHIP (Abeam, Cambridge, Mass.), anti-hsp40 (SPA-450, StressGen, Victoria, BC, Canada), anti-hsp90α (SPA-840, StressGen), antihsp90α (polyclonal, GeneTex, San Antonio, Tex.) anti-p23 (Alexis Biochemicals, San Diego, Calif., 804-023-R100), anti-acetyl-lysine (monoclonal) and anti-AKT (Cell Signaling, Beverly, Mass.), anti-Acetyl lysine (polyclonal, Upstate-Millipore), anti-HA.11 (Monoclonal, Covance, Berkeley, Calif.), anti-cRaf and anti-MMP2 (Santa Cruz Biotech., Santa Cruz, Calif.), anti-FLAG (M2 monoclonal and F polyclonal), ANTI-FLAG® M2 agarose, anti-β-actin (Sigma, St. Louis, Mo.) and agarose conjugates (Upstate, Lake Placid, N.Y.). Plasmids expressing FLAG (F)-hsp90 and HA-p300 have previously been described (Koga, F., et al., Proc Natl Acad Sci USA., 103:11318-22 (2006); Zhao, B. X., et al., EMBO J., 25:5703-15 (2006)).

Acetylated-K69 hsp90α Antibody:

Affinity-purified polyclonal antibody against Ac-K69-hsp90α was generated by Alpha Diagnostic (San Antonio, Tex.) based on the synthetic 12 amino acid peptide flanking K69 (acetylated) ETLTDPSKLDSGK (SEQ ID NO:15). Affinity-purified antibody was checked by performing an ELISA, using free peptide containing acetylated lysine. The antibody specifically recognized acetylated peptide but not non-acetylated peptide dotted on nitrocellulose membrane (data not shown). The antibody also recognized increase in hsp90α acetylation following HDAC inhibitor-treatment and untreated acetylated hsp90α (see FIG. 6B).

Transfections, Immunoprecipitations and Immunoblots:

Following culture in the plates for 24 hours, cells were transfected by Lipofectamine Plus following the protocol provided by Invitrogen (Carlsbad, Calif.). Transfected cells were cultured in full-medium containing drugs dissolved in DMSO or vehicle for 24 hours. Cellular extracts were prepared by directly adding lysis buffer (25 mM Tris-phosphate, pH 7.8, 2 mM DTT, 2 mM 1,2-diaminocyclohexane-N′,N′,N′,N′-tetraacetic acid, 10% glycerol, 0.2% Triton X-100) to the cells on ice. For immuno-precipitations (IP), 2×10⁶ cells in 100 mm dishes were transfected and/or treated as described above. Cellular extracts were prepared and immunoprecipitation performed as described 24. For immunoblotting, cellular extracts or immunoprecipitates were separated on SDS-PAGE, transferred to a nitrocellulose membrane, probed with antibodies, and visualized with enhanced chemiluminescence, as described (Chen, Z., et al., Mol. Pharmacol., 69:1527-33 (2006)).

Purification of Acetylated hsp90α:

Using anti-FLAG M2 affinity beads, F-tagged hsp90α protein was affinity captured from F-hsp90α transfected-HEK 293 cells that had been treated with 100 nM LBH589 (Novartis Pharmaceuticals Inc., East Hanover, N.J.). This was followed by immunoprecipitation of acetylated F-hsp90α using acetyl lysine agarose beads. The immunoprecipitated proteins were resolved by 8% SDS-PAGE gel and visualized using Coomassie Blue stain.

### Results

Hsp90 is acetylated both in vitro and in vivo. The HAT responsible for inducing hyperacetylation of hsp90α was identified. In HEK293 cells with ectopic expression of FLAG (F)-hsp90α, treatment with the pan-HDAC inhibitors LBH589 or vorinostat resulted in hyper-acetylation of F-hsp90α. To determine whether p300 acts as the HAT for hsp90α, following incubation with p300 and acetyl-CoA, in vitro translated hsp90α was analyzed for its acetylation status by Western analysis with anti-AcK antibody. p300 was necessary for the acetylation of hsp90α in the in vitro assay. Immunoprecipitated F-hsp90 from LBH589-treated cells was used as the positive control. In HEK293 cells, it was determined that p300 can be coimmunoprecipitated with hsp90α. Co-incubation with p300 was discovered to dose-dependently stimulate the acetylation of hsp90α. This was not seen with PCAF. However, knock-down of p300 using siRNA only partially decreased LBH589 induced acetylation of hsp90. Therefore, p300 appeared not to be the sole but one of the HATs both for the in vitro and in vivo acetylation of hsp90α.

### Example 2

### Identity and Functional Significance of Lysine Residues in hsp90α Hyper-Acetylated by pan-HDAC Inhibitors

### Methods and Materials

Hsp90 Mutants

Hsp90 mutants were generated by site-directed mutagenesis using the QuikChange® kit from Stratagene (Cedar Creek, Tex.).

The sequence of mutagenesis primers were:

K69Q

(SEQ ID NO: 1)

5-GAA AGC TTG ACA GAT CCC AGT CAA TTA GAC TCT GGG
A
K69R

(SEQ IF NO: 2)

5-GAA AGC TTG ACA GAT CCC AGT AGA TTA GAC TCT GGG
A
K100Q

(SEQ ID NO: 3)

5-GAT ACT GGA ATT GGA ATG ACC CAG GCT GAC TTG ATC
K100R

(SEQ ID NO: 4)

5-GAT ACT GGA ATT GGA ATG ACC AGG GCT GAC TTG ATC
K292Q

(SEQ ID NO: 5)

5-TCG ATC AAG AAG AGC TCA ACC AAA CAA AGC CCA TCT
G
K292R

(SEQ ID NO: 6)

5-TCG ATC AAG AAG AGC TCA ACA GAA CAA AGC CCA TCT
G
K327Q

(SEQ ID NO: 7)

5-TGG GAA GAT CAC TTG GCA GTG CAG CAT TTT TCA GTT
G
K327R

(SEQ ID NO: 8)

5-TGG GAA GAT CAC TTG GCA GTG AGG CAT TTT TCA GTT
G
K478Q

(SEQ ID NO: 9)

5-GTG ATG AGA TGG TTT CTC TCC AGG ACT ACT GCA CCA
G
K478R

(SEQ ID NO: 10)

5-GTG ATG AGA TGG TTT CTC TCA GGG ACT ACT GCA CCA
G
K546Q

(SEQ ID NO: 11)

5-GAA GAC TTT AGT GTC AGT CAC CCA AGA AGG CCT GGA
ACT
K546R

(SEQ ID NO: 12)

5-GAA GAC TTT AGT GTC AGT CAC CAG AGA AGG CCT GGA
ACT
K558

(SEQ ID NO: 13)

5-TCC AGA GGA TGA AGA AGA GCA AAA GAA GCA GGA AG
K558R

(SEQ ID NO: 14)

5-TCC AGA GGA TGA AGA AGA GAG AAA GAA GCA GGA AG

Mass Spectrometric Determination of hsp90α Acetylation Sites:

For comprehensive detection of the acetylation sites, MS in combination with MS/MS was utilized 25. First, gel slices from SDS PAGE separation of cell lysates were subjected to in-gel tryptic digestion to create peptides whose mass can be searched against public data bases. For peptide detection, we used an Applied Biosystems 4700 Proteomics Analyzer with Mascot (Matrix Science) protein search engine. The sample was loaded using α-cyano-4-hydroxycinnamic acid (CHCA) into the instrument according to manufacturer's instructions and run in a data dependent MS plus MS/MS mode. This allows for the documentation of the peptides creating a protein fingerprint for protein identification as well as documentation of the peptides for further analysis through post-source fragmentation. Once the peptides were detected, the instrument was told to go back to each sample and fragment the individual top 20 peptides creating fragments of different lengths that can be re-assembled by the computer to predict, in combination with the identification from the fingerprint data, the sequence of the peptide. The sequence data, in turn was used to bolster the protein identification call. The program allows for certain user input modifications that will allow for missed cleavages as a result of enzyme inefficiency as well as blocked sites. The software also allows for input of potential modifications that might add additional mass to a peptide allowing the search engine to call the peptide in either its modified or unmodified state and with or without missed cleavages resulting from any modifications or enzyme inefficiencies. All of this information was amassed and the call for identification and of the potential modification at a certain site was statistically calculated and the highest probability calls are reported.

ATP-Sepharose Binding Assay:

Hsp90α in 200 μg of cell lysates was affinity-precipitated using KinaseBine™ γ-phosphate-linked ATP resin (Innova Biosciences) at 4° C. for 4 hours. After washing three or four times with the lysis buffer, the resin was pelleted and SDS/PAGE analysis was performed (Bali, P., et al., J Biol Chem., 280:26729-34 (2005)).

Biotinylated-Geldanamycin (B-GA) Binding Assay:

Biotinylated (B)-GA binding to hsp90 was assessed as described previously 26. Briefly, cell-lysates were incubated with or without 17-AAG (Developmental Therapeutics Branch of CTEP/NCI/NIH) for 1 hour at 4° C., and then incubated with B-GA to displace 17-AAG from hsp90 for another 1 h. GM-bound hsp90 was captured by biotin-GA linked to streptavidin Mutein Matrix (Roche Diagnostics, Indianapolis, Ind.) for 1 h at 4° C. The unbound supernatant was removed and the beads were washed three times with lysis buffer. The precipitates were immunoblotted for hsp90.

### Results

The identity of the acetylated lysine residues in hsp90α induced by HDAC inhibitor LBH589 was determined. HEK293 cells transfected with F-hsp90α, were treated with 100 nM LBH589, and the acetylated F-hsp90α was affinity immunopurified using anti-FLAG conjugated M2 agarose, followed by agarose beads bearing immobilized anti-AcK antibody. The enriched acetylated hsp90α was analyzed by nano-HPLC/MS/MS in a mass spectrometer. Seven acetylated lysine residues were identified in hsp90α: K69, K100, K292, K327, K478, K546 and K558. All of the identified lysine residues that are acetylated reside on the surface of the protein and are thus accessible for modification.

To assess the impact of acetylation at the various lysine residues on hsp90α function, point mutations were introduced to create acetylation-deficient (lysine to arginine, K/R) and acetylation mimetic (lysine to glutamine, K/Q) mutants on the FLAG (F)-tagged hsp90α. First, it was determined whether any of the K/R point mutations affects the overall hyperacetylation of F-hsp90α induced by either the co-transfected p300 or by treatment with LBH589. None of the individual K/R mutants showed any change in the hyperacetylation induced by either the co-transfected p300 or by treatment with LBH589. Transfectants of F-hsp90α, with or without K/R substitutions, were treated with or without 100 nM of LBH. Following this, immunoprecipitates with M2 antibody were immunoblotted with either anti-AcK or anti-F antibody.

The mutants were also analyzed for their ability to bind ATP, as well as for binding to co-chaperones, client proteins and the biotinylated (B) GA (geldanamycin) (Kamal, A., et al., Nature., 425:407-10 (2003)). Precipitates from the mixture of cell lysates containing hsp90α and ATP-sepharose were analyzed with anti-F antibody. Although none of the K/R mutants compromised the ATP binding of Fhsp90α, all but one of the acetylation-mimetic mutants (K/Q) showed decreased binding to ATP. Also, following transfections of F-hsp90α, with or without K/Q or K/R substitutions, immunoprecipitates with M2 antibody were immunoblotted with anti-CHIP, anti-p23, anti-hsp40, anti-hsp70, anti-c-Raf or anti-F antibody.

Only the K292Q mutant demonstrated increased binding to ATP. The significance of this is unclear, although K292 is in the hinge region at the beginning of the middle domain (MD) of hsp90α, a region which is well conserved from yeast to human hsp90 (Scroggins, B. T., et al., Mol. Cell., 25:151-9 (2007); Meyer, P., et al., Mol. Cell., 11:647-58 (2003)). Increased acetylation of hsp90 by a pan-HDAC inhibitor or HDAC6 siRNA had been shown to inhibit the binding of hsp90 with co-chaperones, e.g. p23, and client proteins (Bali, P., et al., J Biol Chem., 280:26729-34 (2005)). Here, it was found that acetylation-mimetic mutants (K/Q) of the lysine residues in the middle domain, i.e., K100, K292, K327, K478, K546 and K558, displayed decreased binding with the co-chaperones p23 and to a lesser extent hsp40. While binding of K/Q mutants at K69, K100, K327, K478, K546 and K558 to CHIP was decreased, binding of K/Q mutant at K292 was not affected. Additionally, binding of K/R mutants was similar to the binding of WT hsp90α to CHIP. Acetylation-mimetic mutants of hsp90α also showed disrupted binding to hsp70 and with its client protein c-Raf, except with the K327Q mutant. Binding of hsp70 and c-Raf to K/R mutants appeared to be not significantly altered.

Cell lysates containing hsp90α and ATP-sepharose were also incubated with B-GA followed by streptavidin coated agarose beads and eluted proteins were analyzed with anti-F antibody. Notably, the acetylation-mimetic K/Q mutants showed increased binding to B-GA. This is consistent with the observation that acetylation of the endogenous hsp90α due to treatment with LBH589 was associated with increase in B-GA binding of hsp90α in MDA-MB-468 cells. MB-468 cells cultured in DMEM medium containing 10% FBS were treated with the indicated concentration of LBH589 for 16 hours. Following this, cell lysates were incubated with B-GA followed by streptavidin coated agarose beads and eluted proteins were analyzed with anti-hsp90α antibody. Acetylation and expression level of endogenous hsp90 were detected with anti-AcK and anti-hsp90 antibody, respectively.

MB-468 cells ectopically expressing F-hsp90α were treated with 100 nM of LBH589 for 16 hours. Following this, equal amount of cell lysates were incubated with 0, 5, 10 or 50 nM of 17-AAG for 30 min at 4° C., followed by incubation with B-GA and streptavidin-coated agarose beads. Precipitates from streptavidin coated beads were analyzed with anti-F antibody. LBH589-induced hsp90 acetylation in MDA-MB-468 cells also promoted 17-AAG binding to the acetylated endogenous hsp90, since B-GA binding to hsp90 was reduced by 17-AAG treatment more in those cells exposed to LBH589 as compared to those that were unexposed.

Following treatment with either vehicle or 100 nM of LBH, cell lysates from HEK293 cells expressing F-hsp90 or K/R substitutions were incubated with vehicle or 50 nM of 17-AAG followed by incubation with B-GA and streptavidin coated agarose beads. Precipitates from streptavidin coated beads were analyzed with anti-F antibody. Following treatment with LBH589, each K/R mutant of hsp90α showed increased binding to B-GA which could not be displaced by co-treatment with 17-AAG, suggesting decreased binding of K/R mutants to 17-AAG.

### Example 3

### Hyperacetylation in Extracellular Location of hsp90α

Previous reports have shown that the inducible isoform hsp90α, but not hsp90β, can be secreted and found on the surface of cancer cells, although it lacks the classical signal sequence (Eustace, B. K., et al., Nat Cell Biol., 6:507-14 (2004); Eustace, B. et al., Cell Cycle., 3:1098-1100 (2004)). Serum-starvation, hypoxia, high concentration of glucose, as well as oxidative stress have all been shown to promote the export and extra-cellular location of hsp90α (Eustace, B., et al., Cell Cycle., 3:1098-1100 (2004); Liao, D. F., et al. J Biol Chem., 275:189-96 (2000); Li, W., et al., Embo J., 26:1221-33 (2007)). Consistent with these reports, the findings herein demonstrate that serum-starvation of breast cancer T47D cells promoted secretion and extracellular localization of the endogenous hsp90α, as well as of the ectopically expressed F-hsp90α. Concentrated extra-cellular medium or cell lysates from T47D cells, which were either serum-starved or cultured in 10% FBS, were immunoblotted with anti-hsp90α antibody. β-actin served as a loading control. Under starvation, both endogenous and exogenous hsp90α are secreted from T47D cells in the acetylated form. Extra-cellular hsp90α was immunoprecipitated with anti-AcK antibody and immunoblotted with either anti-hsp90α or M2 antibody. Notably, treatment with LBH589, in a dose-dependent manner, also promoted and increased the export and extracellular localization of hyper-acetylated hsp90α from T47D cells into the culture medium. These data indicate that hyper-acetylation stimulates the extracellular export of hsp90α.

To further verify this, the export and extracellular location of acetylation mimetic (K/Q) or acetylation-resistant (K/R) mutants transfected into T47D cells that were cultured under serum-free condition were determined. In serum-starved T47D cells, K/R substitutions at K69, K100 and K558 decrease, while K/Q substitutions increase the level of extracellular hsp90α. Supernatants of serum starved T47D cells transfected with the F-tagged hsp90α mutant constructs K69Q, K100Q, K292Q, K327Q, K478Q, K546Q, and K558Q were concentrated and immunoblotted with anti-F antibody. Coomassie-stained non-specific proteins served as the loading control. K69Q, K100Q and K558Q substitutions promoted the export and extracellular location of hsp90α, while K/R substitutions at the same residues markedly reduced the export and extracellular location of hsp90α. Cotreatment of cells transfected with K/R mutants with LBH589 increased the export and extracellular location of K/R mutants of hsp90α to a variable extent, with K100R and K558R mutants showing less export than the other mutants.

MB-231 cells transfected with the K69Q, K100Q, K292Q, K327Q, K478Q, K546Q, and K558Q F-tagged K/Q hsp90α mutant constructs were cultured under serum-free condition for 24 hours and followed by the labeling of surface protein with biotinylation. Biotinylated hsp90 on cell surface was detected with anti-F antibody. Biotinylated actin on cell surface served as loading control. Supernatants of serum-starved M13-231 cells transfected with K69Q, K100Q, K292Q, K327Q, K478Q, K546Q, and K558Q F-tagged K/Q hsp90α mutant constructs were also concentrated and immunoprecipitates with anti-M2 conjugated beads were immunoblotted with anti-MMP-2 antibody. Immunoblot analysis of the solubilized biotinylated membrane proteins also showed that acetylation-mimetic mutants K69Q, K100Q, K327Q and K558Q exist more on the cell surface compared to the wild-type hsp90.

### Example 4

### Extracellular Hyperacetylated hsp90α Binds MMP-2 and Promotes Tumor Cell Invasion

### Methods and Materials

In Vitro Invasion Assays;

In vitro invasion assay were performed, as previously described 12, 27 by using the Cultrex® Cell Invasion Assay kit (RandD Systems, Minneapolis, Minn.). In brief, serumstarved cells in 50 μL serum-free medium with or without antibody or IgG were placed in the top chamber and allowed to invade for 24 hours. The lower chambers (assay chamber) were filled with 10% FBS medium. After incubation, migrated cells on the upper chamber of the membrane were dissociated with cell dissociation solution containing Calcein AM at 37° C. for 1 hour and read the bottom plate at 485 nm excitation and 520 nm emission.

In vitro protein acetylation assay. Protein acetylation assays were performed as described previously 28. Briefly, reactions (30 μL) were carried out at 30° C. for 1 hour with M2 beads bound FLAG-hsp90α (25 μL M2 beads mixed with 10 μl of in vitro translated FLAG-hsp90α and washed three times with HAT buffer) and 50 ng of p300 protein (upstate) in HAT buffer (50 mM Tris-HCl, pH 8.0, 10% glycerol, 1 mM DTT, 1 mM PMSF, 0.1 mM EDTA and 50 nM acetyl-CoA (sigma). After three time wash with HAT buffer, the samples were then subjected to western blot analysis with anti-acetyl-lysine antibody.

### Results

In cancer cells, extra-cellular hsp90α was shown to act as a chaperone and assist in the maturation of the matrix metalloproteinase (MMP)-2 to its active form (Eustace, B., et al., Cell Cycle., 3:1098-1100 (2004)). Consistent with this, the data show that K/Q mutants expressed in MB-231 cells, especially K69Q, K100Q and K558Q, which are preferentially extra-cellular under serum-free culture conditions, bind MMP-2 in the extracellular medium (see Example 3). Overall, these data indicate that acetylation promotes not only the extracellular location of hsp90α but also facilitates its chaperone association with MMP-2.

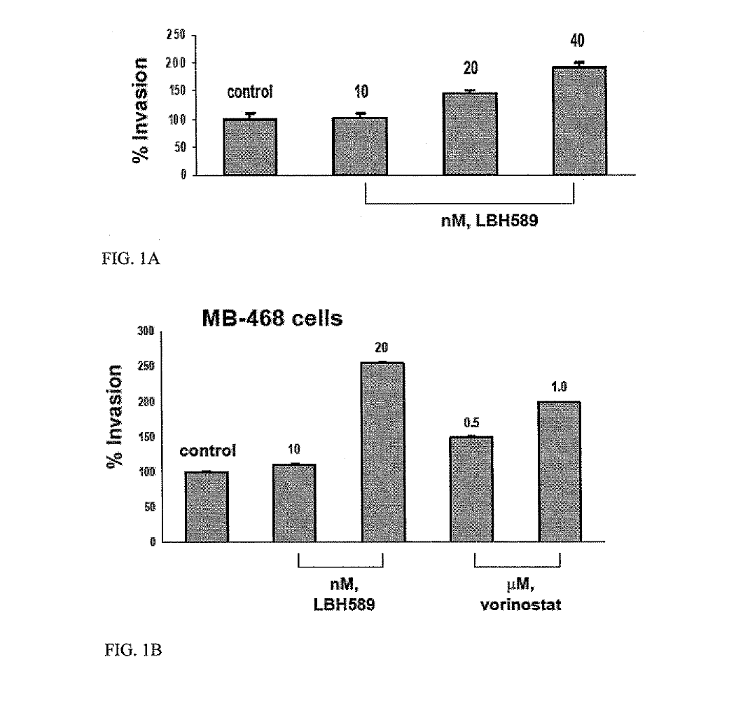

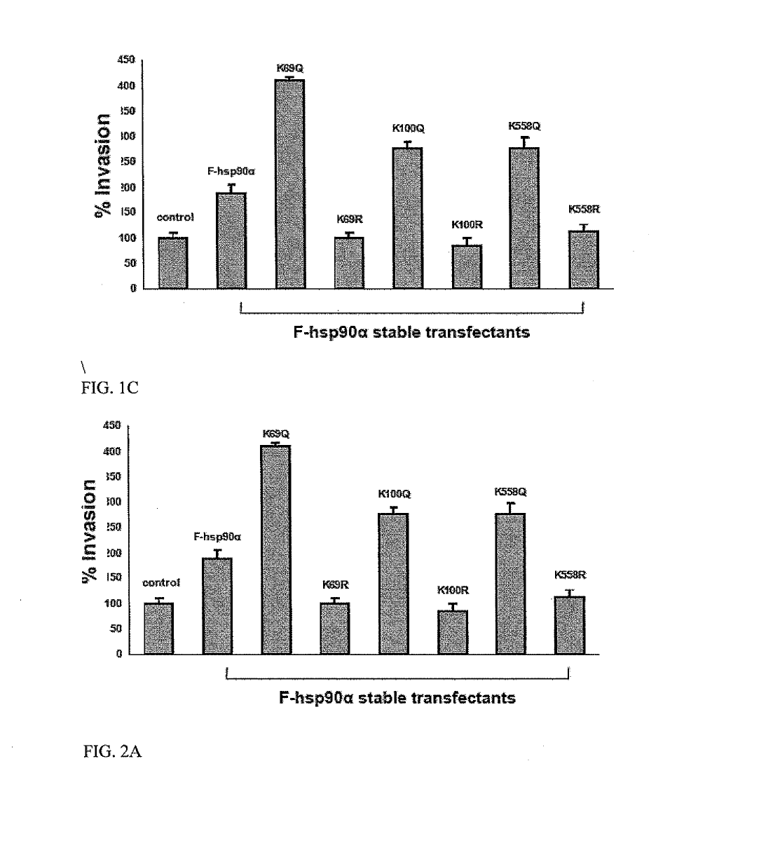

Whether following treatment with sub-lethal concentrations of pan-HDAC-inhibitor increased extra-cellular levels of acetylated hsp90α increases invasiveness of cancer cells was investigated (FIG. 1A). Treatment with LBH589 dose dependently increased matrigel invasion by MB-231 cells. Supernatant of serum starved LBH treated MDA-MB-231 cells was concentrated and immunoprecipitates with anti-hsp90α antibody were immunoblotted with anti-AcK, anti-MMP-2 or hsp90α antibody. Thus, treatment with LBH589 was associated with increased extra-cellular binding of acetylated hsp90α with MMP-2. Similar increase in the invasiveness of MB-468 cells was observed following treatment with LBH589 or SAHA (FIG. 1B). Whether increased extracellular location of K69Q, K100Q or K558Q mutant promotes in vitro invasiveness of breast cancer cells was determined. For this, stable transfectants of MB-468 cells expressing the wild-type hsp90α we created, or the K/Q or K/R mutants of hsp90α at the residues K69, K100 and K558, and their in vitro invasiveness in the matrigel-based assay was evaluated. As compared to the MB-468 cells expressing the wild-type hsp90α, stable transfectants of MB-468 cells with K/Q but not K/R mutants at K69, K100 and K558 demonstrated increased in vitro invasiveness (FIG. 1C).

### Example 5

### Treatment with Anti-AcK hsp90α Antibody Inhibits In Vitro Invasion by Breast Cancer Cells

### Methods and Materials

Confocal Microscopy:

MDA-MB-231 cells were cultured in a chamber slide in RPMI medium with 10% FBS or under serum-free conditions with or without 40 nM LBH589 for 16 hours and stained with anti-acetyl (Ac)-K69 antibody. Briefly, after 16 hours incubation, cells were washed with PBS and fixed with 4% paraformaldehyde for 10 minutes. Following this, the slides were blocked with 3% BSA for 30 minutes and incubated with primary antibody at a dilution of 1:100 in blocking buffer for 2 hours. Following three washes with PBS the slides were incubated in Alexa Fluor 488 anti-rabbit secondary antibody (Molecular probes, Invitrogen) for one hour at 1:3000 dilution. After three washes with PBS, the cells were counterstained with DAPI using Vectashield mountant with DAPI and imaged using Zeiss LSM510 confocal microscope.

### Results

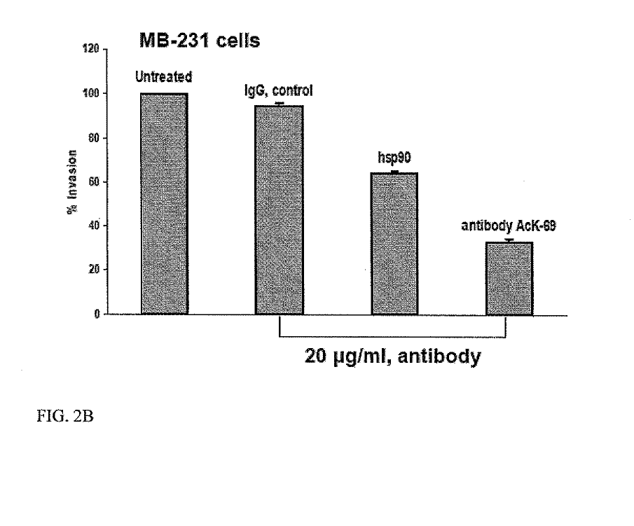

The findings above raised the possibility that treatment with an antibody to acetylated (Ac) hsp90α would inhibit binding of hsp90α to MMP-2 that is involved in tumor invasiveness. Therefore, the anti-Ac-hsp90α would thereby inhibit invasiveness of breast cancer cells. To verify this, the extracellular location of the ectopically expressed K69Q mutant versus the wild-type hsp90α in the breast cancer MB-231 cells was compared. MB-231 cells transfected with either F-hsp90 or K69Q mutant were cultured under serum-free condition for 24 hours. Total extracellular and intracellular hsp90α were immunoprecipitated with anti-M2 conjugated beads and immunoblotted with anti-F antibody. The intensity of the bands was quantified using ImageQuant 5.2 software, and is represented as bar graphs. (FIG. 2A) Compared to the wild type hsp90α, relatively more of the K69Q mutant was extracellular in location. Thus, the K/Q substitution at K69 promotes extracellular location of hsp90α.

Next, a polyclonal antibody was generated against the acetylated K69-containing peptide of hsp90α (anti-Ac-K69). The specificity of the antibody was confirmed by determining its ability to detect the increase in the acetylation of the ectopically expressed endogenous hsp90α following treatment with the HDAC inhibitor LBH589. MB-231 cells were transfected with F-hsp90α followed by the treatment with LBH589. Immunoprecipitates of F-hsp90α with anti-M2 conjugated beads were immunoblotted with anti-K69-hsp90α antibody for the acetylation status and anti-F antibody for F-hsp90α expression. Cells transfected with empty vector followed the treatment with LBH589 served as control for specificity. Acetylation of endogenous hsp90αinduced by LBH589 was also detected with anti-AcK69-hsp90α antibody. Immunoprecipitates of endogenous hsp90α with anti-hsp90α antibody from cell lysates of MB-231 cells treated with or without LBH589 were immunoblotted with either anti-AcK69-hsp90α or anti-hsp90α (rabbit) antibody. IgG served as the control for specificity of the immunoprecipitates.

Following treatment of cells with LBH589, the anti-Ac-K69 hsp90α antibody recognized the increase in the acetylated hsp90α. Importantly, the anti-Ac-K69 hsp90α antibody selectively recognized acetylated hsp90α. In contrast, the commercially available polyclonal anti-hsp90α antibody non-specifically recognized both the acetylated and non-acetylated hsp90α, without showing specific increase in the epitope detection following treatment of the cells with serum-starved condition or with LBH589. Therefore, this raised the possibility that the anti-Ac-K69 antibody might be more selective in attenuating the role of Ac-hsp90α in the invasiveness of cancer cells. Compared to the control IgG-treated or untreated MB-231 cells, serum-starved MB-231 cells demonstrated surface location of the acetylated hsp90α when stained with the anti-Ac-K69 antibody.

Additionally, treatment of serum-starved MB-231 cells with LBH589 led to increased levels of the membrane associated acetylated hsp90α, as detected by the anti-Ac-K69 antibody. Serum-starved MB-231 cells were treated with 40 nM LBH589 for 16 hours, followed by staining with anti-AcK69 hsp90α antibody and confocal microscopy. Cells cultured in RPMI with 10% FBS and cells stained with rabbit IgG served as controls.

The effect of anti-hsp90α with anti-Ac-K69 hsp90α antibody on the in vitro invasiveness of MB-231 breast cancer cells was compared. Serum-starved MB-231 cells treated with 20 μg/mL anti-hsp90α or anti-AcK69 hsp90α antibody were used for determining in vitro matrigel invasion (see Example 4 for protocol). Untreated cells, or cells treated with IgG were used as controls. FIG. 2B clearly demonstrates that, while the control IgG had no significant effect and the commercially available polyclonal anti-hsp90α antibody only modestly inhibited the in vitro invasiveness of MB-231 cells, treatment with the anti-Ac-K69 hsp90α markedly inhibited the matrigel invasion by MB-231 cells (FIG. 2B).

Serum-starved MB-231 cells treated with 20 μg/mL anti-hsp90α or anti-AcK69 hsp90α antibody were used for determining in vitro matrigel invasion (see Example 4). Untreated cells, or cells treated with IgG were used as controls. In vitro invasion by MB-231 cells was inhibited by anti-AcK69 hsp90α antibody. Thus, acetylation of K69 in hsp90α may play an important role in extra-cellular location and chaperone association of hsp90α with MMP-2. Additionally, exposure to anti-Ac-K69 hsp90α antibody could inhibit invasion of breast cancer cells.

### Example 7

### p300 Binds In Vivo to hsp90α

HEK293 cells were transfected with the combination of F-hsp90α and HA-tagged p300, and immunoprecipitates with anti-HA.11 antibody were immunoblotted with anti-F antibody. Cell lysates were also immunoblotted with anti-F, anti-HA.11 or β-actin antibody. Bands were observed in lanes immunoprecipitated with anti-hA.11 antibody and blotted with anti-F antibody indicating that p300 binds to hsp90α in vivo.

Whether p300 acts as one of the HATs for hsp90 in vivo was also investigated. HEK293 cells were transfected with F-hsp90α and scrambled oligo or siRNA against p300, followed by treatment with or without 100 nM LBH589. Immunoprecipitates with anti-AcK from cell lysates were immunoblotted with anti-F antibody. F-hsp90α expression level and endogenous expression level of p300 were detected with anti-M2 and anti-p300 antibody, respectively. B-Actin served as loading control. The data show that b300 acts as one of the HATs for hsp90 in vivo.

### Example 8

### Individual K/R Substitutions do not Affect the Overall p300 or LBH-Mediated Acetylation of F-hsp90α

Transfectants of F-hsp90α (3 μg), with or without K/R substitutions, were co-transfected with HA-tagged p300 (4.5 μg). Following this, immunoprecipitates with M2 antibody were immunoblotted with anti-AcK or anti-F antibody (see Example 1 for protocol). Cell lysates were also immunoblotted with anti-HA or anti-β-actin antibody. The data show that individual K/R substitutions do not affect the overall p300 LBH-mediated acetylation of F-hsp90α.

### Example 9

### Secretion of hsp90α from T47D Cells Promoted by HDAC Inhibitor LBH589 Correlates with the Acetylation of hsp90α in a Dose Dependent Manner

Serum-starved T47D cells were treated with 0, 25, 50, or 100 nM LBH589 [FOR HOW LONG?]. Following this, immunoprecipitates with anti-hsp90α antibody were immunoblotted with anti-AcK or anti-hsp90α antibody. The signal from the immunoblots increased as the concentration of LBH589 increased indicating that secretion of acetylated hsp90α promoted by LBH590 occurs in a dose dependent manner with the concentration of LBH589. Treatment with LBH589 was also found to promote extracellular localization of K/R mutants of hsp90α. Supernatants of serum starved T47D cells transfected with the K69Q, K100Q, K292Q, K327Q, K478Q, K546Q, and K558Q F-tagged K/Q hsp90α mutant constructs were concentrated and immunoblotted with anti-F antibody. The signal from wildtype F-hsp90α transfectants treated with LBH was similar to the signal obtained from

### Example 10

### Anti-AcK69 hsp90α Antibody Selectively Recognizes Acetylated hsp90α in MB-231 Cells

MB-231 cells cultured under 10% FBS or serum-free condition or serum-free plus 40 nM of LBH589 for 16 hours were immunostained with anti-AcK69 hsp90α antibody or anti-rabbit hsp90α antibody, followed by confocal microscopy (see Example 5 for protocol). Rabbit IgG served as control for the specific antibody staining. The data show that anti-AcK69 hsp90α antibody selectively recognizes acetylated hsp90α in MB-231 cells.

1. A pharmaceutical composition comprising an effective amount of a heat shock protein antagonist to inhibit or reduce tumor cell invasion or metastasis, wherein the heat shock protein antagonist specifically binds to acetylated heat shock proteins.

2. The pharmaceutical composition of claim 1 wherein the heat shock protein antagonist comprises an antibody or heat shock protein binding fragment thereof.

3. The pharmaceutical composition of claim 1 wherein the antibody or antibody fragment is chimeric, humanized, single chain, polyclonal, monoclonal, or a diabody.

4. The pharmaceutical composition of claim 1 wherein the heat shock protein is selected from the group consisting of hsp90α and hsp70.

5. The pharmaceutical composition of claim 1 wherein the heat shock protein antagonist is an aptamer or polypeptide.

6. The pharmaceutical composition of claim 1 wherein the heat shock protein antagonist does not bind to the heat shock protein's ATP binding domain.

7. The use of the pharmaceutical composition of claim 1 for the treatment of cancer.

8. A pharmaceutical composition for treating cancer comprising an effective amount of a heat shock protein deacetylase to inhibit or reduce tumor cell invasion or metastasis by inhibiting or reducing heat shock protein acetylation relative to a control.

9. The pharmaceutical composition of claim 8 wherein acetylation of hsp90α or hsp70 is reduced relative to a control.

10. The pharmaceutical composition of claim 8 wherein the heat shock protein deacetylase comprises histone deacetylase.

11. The pharmaceutical composition of claim 8 wherein the cancer being treated is selected from the group consisting of bladder, brain, breast, cervical, colorectal, esophageal, kidney, liver, lung, nasopharangeal, pancreatic, prostate, skin, stomach, uterine, ovarian, and testicular.

12. A pharmaceutical composition comprising an effective amount of a heat shock protein acetylation inhibitor to inhibit or reduce secretion of heat shock proteins in tumor cells or to inhibit or reduce relocation of cytosolic heat shock proteins to the exterior surface of the tumor cells relative to a control.

13. The pharmaceutical composition of claim 11 formulated for local administration, topical administration, oral administration, or parenteral administration.

14. Use of a heat shock protein acetylation inhibitor to inhibit tumor invasion or metastasis.

15. The use of claim 14 wherein acetylation of hsp90α is inhibited or reduce relative to a control.

16. The use of claim 14 wherein acetylation of hsp70 is inhibited or reduce relative to a control.

17. A method for inhibiting or reducing secretion of heat shock proteins by a cell comprising delivering a composition that inhibits acetylation of the heat shock proteins to the interior of the cell.

18. The method of claim 17 wherein the heat shock proteins are selected from the group consisting of Hsp90α and Hsp70.

19. The method of claim 18 wherein cell is a tumor cell.

20. The method of claim 19 wherein the cell is a cancer cell.

21. The method of claim 20 wherein the cancer is selected from the group consisting of bladder, brain, breast, cervical, colorectal, esophageal, kidney, liver, lung, nasopharangeal, pancreatic, prostate, skin, stomach, uterine, ovarian, and testicular.

22. A method for inhibiting tumor cell invasion in a subject comprising administering to the subject a composition that binds specifically to an acetylated amino acid of heat shock proteins, wherein the acetylated heat shock proteins are extracellular or on the tumor cell surface.

23. The method of claim 1 wherein the heat shock proteins are selected from the group consisting of Hsp90α and Hsp70.

24. The method of claim 22 wherein the composition that specifically binds an acetylated amino acid of the heat shock proteins is selected from the group consisting of a polypeptide, an aptamer, and an antibody or antigen binding fragment thereof.

25. The method of claim 24 wherein the antibody or antigen binding fragment thereof is chimeric, humanized, single chain, polyclonal, monoclonal, or a diabody.
